(19) United States
(12) Patent Application Publication
(10) Pub. No.: US 2025/0339382 A1
(43) Pub. Date: Nov. 6, 2025

(54) CORRALLING AMPHIPATHIC PEPTIDE COLLOIDS

(71) Applicant: KANSAS STATE UNIVERSITY RESEARCH FOUNDATION, Manhattan, KS (US)

(72) Inventors: John M. TOMICH, Manhattan, KS (US); Susan K. WHITAKER, Manhattan, KS (US); Sheila M. DE MELLO BARROS, Manhattan, KS (US)

(21) Appl. No.: 18/860,868

(22) PCT Filed: Apr. 28, 2023
(86) PCT No.: PCT/US2023/066373

§ 371 Date: Oct. 28, 2024

Related U.S. Application Data
(60) Provisional application No. 63/335,847, filed on Apr. 28, 2022.

(51) Int. Cl. A61K 9/51; A61K 9/19

(52) U.S. Cl. CPC A61K 9/5169; A61K 9/19; A61K 9/5123; A61K 9/5192

(57) ABSTRACT

Peptide-based colloidal particles that are able to encapsulate and stabilize non-polar excipients, including room temperature solid lipids, and hydrophobic and/or poorly water-soluble active agents for storage and delivery in aqueous mediums, methods of making and using the same. Methods of customizing and resizing such colloids. Peptide-based colloidal particles as delivery vehicles for various hydrophobic and/or poorly water-soluble active agents.

### CROSS-REFERENCE TO RELATED APPLICATIONS

The present application claims the priority benefit of U.S. Provisional Patent Application Ser. No. 63/335,847, filed Apr. 28, 2022, entitled CORRALLING AMPHIPATHIC PEPTIDE COLLOIDS, incorporated by reference in its entirety herein.

### SEQUENCE LISTING

The following application contains a sequence listing submitted electronically as a Standard ST.26 compliant XML file entitled “SequenceListing 57229.xml,” created on Apr. 27, 2023, as 40,960 bytes. The content of the XML file is hereby incorporated by reference.

### BACKGROUND OF THE INVENTION

### Field of the Invention

The present invention relates to formulations and methods for stabilizing non-polar compounds and excipients for delivery in aqueous systems.

### Description of Related Art

There has been a continuing search for methods and compositions which effectively and efficiently deliver lipids or oils, as well as hydrophobic active agents, into an aqueous medium. Conventional approaches for preparing formulations containing hydrophobic active agents have a number of drawbacks, including large structures that agglomerate and eventually lead to complete phase separation. Due to their physical instability and lack of homogeneity, these formulations also suffer from poor and variable cellular absorption. There remains a need for improved technologies for formulating and delivering hydrophobic or poorly water-soluble active agents.

### SUMMARY OF THE INVENTION

The present disclosure is broadly concerned with compositions of colloidal particles, each comprising a peptide layer encapsulating a droplet of non-polar excipient, such as a lipid, oil, grease, or non-polar solvent, optionally along with one or more hydrophobic and/or poorly water-soluble active agents dispersed or distributed therein. Described herein are methods of controlled sizing and re-sizing of the colloidal particles, as well as methods for lyophilization and rehydration of the colloidal particles. Described herein are also new peptide sequences for preparing the colloidal particles, as well as new techniques for encapsulating and stabilizing room temperature solid lipids, oils, and fats in the colloidal particles (e.g., those lipids in solid state at room and body temperature, usually long chain triglycerides or partial glycerides, etc.).

Also described herein are methods for delivering hydrophobic and/or poorly soluble active agents to a subject in need thereof. The methods comprise administering a composition according to various embodiments described herein to the subject.

The application also concerns methods for delivering hydrophobic and/or poorly soluble active agents to plants. The methods comprise applying a composition according to various embodiments described herein to at least a portion of a plant and/or to the soil where a plant is or will be planted. In some embodiments, the active agents are applied to the plant and/or to the soil where a plant is or will be planted for the purpose of delivery of the active agent to an insect pest.

Also described herein are methods for delivering hydrophobic and/or poorly soluble active agents, specifically insecticides, to insects. The methods comprise contacting the insect with a composition according to various embodiments described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Figure (FIG. 1 is a cartoon illustration of a colloidal particle according to an embodiment of the invention, including an enlarged view of the peptide monolayer that coats and stabilizes the non-polar excipient droplet.

FIG. 2 shows circular dichroism (CD) spectra of CAPC peptides encapsulating different solvents or oils.

FIG. 3 shows CD spectra of different CAPC peptide sequences used to encapsulate soy oil. The C- and N-terminal protecting groups are not included in the legend.

FIG. 4 shows mass spectra from trypsin digest of free and aggregated CAPC peptides for A. intact control CAPCs; B. CAPCs after 1 hour; and C. free aggregated CAPC peptide digested for 1 hour.

FIG. 5A shows graphs from NTA analysis of the colloidal particle mixture right after sonication. The tracings to the right of the distribution curves are the individual data sets generated for the readings the instrument recorded.

FIG. 5B shows graphs from NTA analysis of the colloidal particle mixture after resizing. The tracings to the right of the distribution curves are the individual data sets generated for the readings the instrument recorded.

FIG. 6 is a bar graph of particle size and temperature stability of CAPC colloidal particles containing coconut oil prepared at 37° C. (n=3).

FIG. 7 is a graph of differential scanning calorimetry analysis of, peptides prepared with no oil (h5L, smaller dashes); coconut oil (CCO, larger dashes); and CAPC colloidal particles prepared using the same peptide sequence with coconut oil (CAPCs, solid line).

FIG. 8 shows confocal microscope images of A. Green CF-labeled peptide in water (reference bar 10 microns); B. soy oil containing Nile red (reference bar 20 microns); and C. CAPC colloidal particles forming a green monolayer containing soy oil with Nile red (reference bar 5 microns).

FIG. 9 is a transmission electron micrograph (TEM) image of resized methyl mercury labeled CAPC.

FIG. 10 shows confocal microscope images of dried/rehydrated CF-labeled CAPC encasing soy oil containing Nile red, where A. image of the wavelength showing just CF-labeled peptide; B. image of the wavelength that detects soy oil containing Nile red; and C. image of merged images. Reference bar set at 2 microns.

FIG. 11 shows imaging of in vitro cellular uptake by CHO cells of CF-CAPC colloidal particles containing soy oil with Nile red. The bottom left image is a bright field image and the bottom center image is a merged picture showing all of the different fluorescent components. The one on the bottom right is a blank for reference.

FIG. 12 is a graph showing the effect of sonication time on colloidal particle size and distribution density.

FIG. 13 is a graph of the results from oral dosing of dogs with CBD nanoformulated CAPCs (NANO) or MCT oil as the control (MCT).

### DETAILED DESCRIPTION

The present disclosure is concerned with peptide-based colloidal particles that are able to encapsulate and stabilize non-polar compounds for storage and delivery in aqueous mediums. For example, the peptides can be used to encapsulate and stabilize droplets or particles of lipids, oils, greases, fats, and non-polar solvents in a hydrophobic core surrounded by a peptide monolayer. The hydrophobic core advantageously sequesters a non-polar excipient, such as a lipid, oil, or non-polar solvent, optionally along with one or more hydrophobic and/or poorly soluble active agents dispersed or distributed therein, into discrete colloidal particles that can remain stably suspended or dispersed in an aqueous medium.

In more detail, we disclose improvements in the use of linear peptides that are able to encapsulate or form a coating around droplets or particles of lipid oils and other hydrophobic or poorly (water) soluble active ingredients, allowing them to disperse as stable colloidal particles suspended in water. The cationic outer surface of these particles is hydrophilic, allowing them to disperse in aqueous solutions, and fostering uptake by animal and plant cells and tissues thereby facilitating the delivery of lipid-soluble active ingredients to the interior of cells. Other carriers, adjuvants, synergists, dispersing agents, or solutions may also be included within/with the particles. The particles also shield the active agent from the external environment, which could prematurely inactivate the active agent. As drug delivery vehicles, the novel colloidal particles can also be used to alter the biological half-life of an active ingredient.

The present invention is broadly concerned with compositions comprising a plurality of colloidal particles suspended in an aqueous carrier. The colloidal particles each comprise a peptide layer or coating with a cationic, hydrophilic exterior surface within which is sequestered a non-polar excipient, such as a lipid, oil, or non-polar solvent, optionally along with one or more hydrophobic and/or poorly soluble active agents dispersed or distributed therein. The particle is characterized by a cationic, hydrophilic outer surface formed of the C-terminal hydrophilic segment of the peptides orienting outward towards the external environment in each particle. The inward facing surface of the peptide layer is hydrophobic formed of the N-terminal hydrophobic segment of the peptides orienting towards the internal hydrophobic core of the particles. Preferably, the peptide layer or coating is homogenous, meaning that it is comprised of a plurality of the same type of peptide (i.e., peptides having the same amino acid sequence)

The peptide sequences used to prepare the peptide coating or layer are amphipathic and linear with no branch point, comprising (consisting essentially, or consisting of) an N-terminal hydrophobic segment (first terminal end) and a C-terminal hydrophilic segment (second terminal end). The peptides preferably have a molecular weight ranging from about 550 Da to about 2300 Da, and more preferably from about 675 Da to about 2050 Da, and even more preferably from about 800 Da to about 1800 Da. The “molecular weight” for these peptides is an average weight calculated based upon the total MW of the actual coupled amino acids present divided by the number of residues. The linear peptides have an overall chain length ranging from 20 amino acid residues or less in length, preferably from about 5 to about 20, more preferably from about 8 to about 15 residues in length, and even more preferably from about 8 to about 12 residues in length. Peptides can be synthesized using traditional Fmoc chemistries.

The N-terminal hydrophobic head groups are preferably each from about 3 residues to about 11 residues in length, and more preferably from about 4 to about 10 residues in length, and even more preferably from about 5 to about 9 residues in length. Amino acids used for the N-terminal hydrophobic segment are preferably selected from hydrophobic or very hydrophobic residues, such as leucine, isoleucine, valine, phenylalanine, and methionine. In one or more embodiments, the N-terminal hydrophobic segment can include up to two neutral amino acid resides selected from glycine, serine, and/or threonine. In one or more embodiments, the N-terminal hydrophobic segment is free of alanine residues. Particularly preferred hydrophobic amino acids for use in the hydrophobic segment include phenylalanine, leucine, isoleucine, and valine. If present, the neutral amino acid residues are preferably selected from glycine and/or serine. In one or more embodiments, the hydrophobic segment comprises a sequence XLIVI (SEQ ID NO: 1), XLIVIGSII (SEQ ID NO:2), XFFIVIL (SEQ ID NO:3), or XLIVIGSIIVIL (SEQ ID NO: 4), where X is F or V, and where the amino acid residues can be in order or in any order (scrambled, see e.g., SEQ ID NOs: 14-32). In one or more embodiments, the N-terminal hydrophobic segment comprises a sequence X (LIVI) (SEQ ID NO:1), X (LIVI) GSII (SEQ ID NO: 2), XFF (IVI) L (SEQ ID NO:2), or X (LIVI) GSIIVIL (SEQ ID NO:4), where X is F or V, and where the residues in parentheses are in order or are in any order (scrambled). In one or more embodiments, the residues in parentheses are replaced with all I residues or all V residues. In one or more embodiments, any one of the residues in the sequences FLIVI (SEQ ID NO:1), FLIVIGSII (SEQ ID NO:2), VFFIVIL (SEQ ID NO:3), or FLIVIGSIIVIL (SEQ ID NO:4), except for the N-terminal phenylalanine can be replaced with an I or V.

The C-terminal hydrophilic (polar) tail segment preferably comprises from about 1 to about 7 hydrophilic and cationic amino acid residues (each), preferably lysine, but may include histidine, arginine, aspartic acid, or glutamic acid, which also have electrically charged side chains. In one or more embodiments, the hydrophilic tail segment is free of any arginine residues (preferably the entire peptide is free of any arginine residues). More preferably, the C-terminal hydrophilic tail consists of lysine residues, more preferably from about 1 to about 6 lysine residues, and even more preferably from about 1 to about 5 lysine residues. A particularly preferred lysine sequence is KKKKK (SEQ ID NO:5).

In one or more embodiments, the peptides comprise an added cysteine residue at the C-terminus of the peptide, preferably connected at the terminal lysine position, to facilitate further functionalization. In some embodiments, the N-terminal end of each hydrophobic segment can be capped with an acetyl group (Ac).

Exemplary peptides are also described in PCT/US2020/023891, filed Mar. 20, 2020, and published as WO 2020/198020, incorporated by reference in its entirety herein. Now termed Corralling Amphipathic Peptide Colloids (CAPCs), these linear peptides do not form bilayers or micelles but rather when mixed with a hydrophobic composition, turn lipid solutions into encapsulated colloids of lipid droplets or particles, with a cationic surface that are readily taken up by cells. Hydrophobic active ingredients soluble in lipids, oils, and non-polar solvents can be delivered using these colloids. Further, these peptides are able to capture nearly 100% of active ingredients dissolved in the hydrophobic phase.

In one or more embodiments, functional groups and/or various moieties can be attached to the C-terminal lysine, or the C-terminal carboxyl group, or in the case of a C-terminal cysteine, the free sulfhydryl group. In this way, the peptides can be modified with a variety of targeting moieties, which will locate on the outside of the colloid and can be used for targeting, detectable labeling (e.g., fluorescent labels), and the like. For example, the peptides can be iodinated for targeting. The term “functional moiety” is used herein to encompass functional groups, targeting moieties, and active agents that may be attached to the outer surface of the particle. Exemplary functional moieties that can be attached include fluorophores, dyes, tissue targeting moieties and ligands, antibodies, cysteine, cysteamine, biotin, biocytin, nucleic acids, polyethylene glycol (PEG), organometallic compounds, (e.g., methyl mercury), radioactive labels, conjugating chemistries, —COOH, —NH₃, —SH and the like. Multiple such moieties can also be attached in a chain of sequential order from the C-terminal end using aliphatic spacers to separate different moieties. Thus, the invention provides the opportunity to create multi-functionalized colloidal particles. Since the individually modified peptides self-assemble to form the matrix, any number of functional moieties at different stoichiometries can be adducted onto individual peptide sequences that comprise part of the assembled colloidal particle.

FIG. 1 provides an illustration of a colloidal particle according to an embodiment of the invention. In the figure, the hydrophobic droplet or particle is encapsulated or encased by a layer of peptide. As shown in the enlarged depiction, the squiggled lines represent the amphipathic peptides. The peptides form the peptide membrane or layer, with their cationic hydrophilic residues facing the aqueous external environment and the hydrophobic residues extending towards the interior/core of the colloid and interacting with the lipid, oil, or non-polar solvent molecules, such that the peptides assemble to form a monolayer at the oil-water interface, corralling the lipid, oil, or non-polar solvent into discrete particles. Importantly, however, the peptides themselves are not conjugated or otherwise bound to the active agents or hydrophobic core materials.

Advantageously, however, the peptide layer interacts with hydrophobic droplets to form a monolayer that stabilizes the encapsulated hydrophobic material, such that the colloidal particles remain stable, as discrete colloidal particles, in an aqueous solution for extended periods of time, without agglomeration, coalescing, or falling apart (preferably for at least 3 months, more preferably at least 6 months, even more preferably at least 12 months). Advantageously, the colloidal particles are stable in suspension and do not fuse over time into larger aggregates or larger colloidal particles. This is referred to herein as the “shelf life” or “shelf stability” of the colloids. In particular, the present report details the advantageous shelf-stability of the colloids. For example, the formed colloids exhibit shelf-stability at ambient conditions when stored in an aqueous solution (e.g., water) for more than 400 days.

Particular improvements to the technology as reported herein include controlled size distribution of the colloids as well as techniques for re-sizing the colloids. For example, as reported herein, the size of the colloids can be adjusted by modifying the temperature of the reaction solution, e.g., where the colloid average sizes are reduced in colder temperatures.

Approaches for lyophilization or spray-drying of the colloids and subsequent rehydration are also described, confirming that the hydrophobic material remains encapsulated in the peptide monolayer, which does not break apart during lyophilization, and can be reconstituted via hydration of the lyophilized powder in aqueous medium.

The colloidal particles are prepared by mixing the lipid, oil, fat, grease, or non-polar solvent (i.e., excipient) with peptide in a reaction vessel. In one or more embodiments, an active agent is first dispersed or dissolved in the hydrophobic bulk excipient. Preferred lipids, fat, and oils are vegetable oils (coconut, soy, avocado, etc.), mineral oils, migloyls oils, paraffin oils, Solutol®, and the like, or combinations thereof. The oil may itself be an active in its own right, or it may contain actives. Preferred non-polar or low dielectric solvents (i.e., those having a low dielectric constant) include essentially any solvent that is immiscible with water. Water has a dielectric constant at room temperature (˜25° C.) of about 78.2. Exemplary solvents include those with a dielectric constant at room temperature (˜25° C.) of less than 50, preferably less than 30. Examples include alkanes (pentane, hexane, heptane, and n-Decane), cycloalkanes (cyclohexane), diethyl ether, carbon tetrachloride, methylene chloride, aromatics (benzene, toluene, and xylene), phthalate esters (diethyl phthalate), piperonyl butoxide, and the like, or combinations thereof.

Preferably, the active agent, if any, is first dissolved, suspended, or dispersed in the bulk excipient. The peptide is added in sufficient quantity to encase all of the excipient present, mixed with the excipient, and then allowed to stand for at least about 15 minutes, preferably from about 15 minutes to about 30 minutes. In one or more embodiments, peptide is added at a concentration of from about 0.5 mM to about 5 mM, preferably from about 1 mM to about 3 mM. In one or more embodiments, the weight ratio of peptide to excipient is from about 1:50 to about 1:20, preferably from about 1:25 to about 1:10. Water (preferably distilled/deionized) or other aqueous solvent system is then added, preferably in excess, and the resulting emulsion is mixed or agitated to uniformly distribute or suspend the (otherwise immiscible excipient) in the aqueous solvent system. More preferably, the mixture is mixed or agitated using a vortex mixer or bath sonicator for at least about 5 minutes, preferably from about 5 minutes to about 15 minutes. The homogenously or uniformly mixed composition becomes somewhat cloudy as the colloids form with the peptide encapsulating particles or droplets of the excipient and stabilizing them in the aqueous solvent system, such that they become suspended and distributed throughout the aqueous solvent system. Upon centrifugation, the colloids move to the top of the water column, and notably, no more oil layer is visible. As shown in FIG. 1, the hydrophobic amino acids in the peptide sequence point towards the interior of the colloid and interact with the bulk excipient droplet that has been encased by the peptide monolayer.

Moreover, as described herein, the colloids can advantageously be used to encapsulate lipids, fats, grease, and oils that are room temperature solids. For example, the peptides can be dispersed into a solution containing a lipid, fat, grease, or oil under conditions above their melting temperatures such that the melted lipid, fat, grease, or oil is in the liquid state. Once the colloids form around the melted lipid, fat, grease, or oil, the colloidal suspension can be returned to room temperature whereupon the encapsulated lipid, fat, grease, or oil is solidified with a peptide coating or layer stabilizing and protecting the lipid, fat, grease, or oil payload. Coconut oil and stearate-based glycerol, triglycerides (tri-stearin), partial glycerides (Imwitor), fatty acids (stearic acid, palmitic acid), steroids (cholesterol), and waxes (cetyl palmitate) are examples of such higher melting lipids.

In one or more embodiments, the colloidal particles have a maximum surface-to-surface dimension (e.g., the diameter of a substantially spherical particle) of greater than about 25 nm, preferably from about 100 nm to about 5 microns, more preferably from about 200 to about 1,000 nm. For ease of reference, the terms “diameter” or “particle size” are used interchangeably herein to refer to the maximum surface-to-surface dimension of each particle. Moreover, since the methods of the invention yield suspensions of a plurality of particles, the “particle size” referenced herein may refer to the average (mathematical mean) diameter of the entire population of particles in the suspension.

The particles can be resized (or reduced in size) if desired, such as by extruding the composition through any combination of filters, small bore conduits, such as hollow syringes, and the like having an open bore or pore size of the desired size. For example, it is commonly desired to have particles of a size of 200 nm or less to improve cellular uptake. The colloidal particles can be resized, for example, by passing through a combination of syringes and/or filters, whereby the larger colloidal particles are blebbed or pinched off into smaller colloidal particles. In other words, the larger colloidal particles will split apart into smaller particles, such that the suspension before resizing has the same volume, but contains more particles after resizing. As discussed here, sizing can also be controlled during the fabrication process by reducing the temperature to about 4° C. to reduce the size of the formed colloidal particles. However, it will also be appreciated that larger sized colloids may be useful to accommodate the electrostatic binding of much larger oligonucleotides to their surface for delivery, ranging up to 10,000 bases.

Advantageously, the colloidal particle has a low polydispersity, with a PDI of less than 250%, preferably less than 100%, more preferably less than 50%, more preferably less than 40%, even preferably from about 2% to about 30%. Another important aspect of the design of the colloidal particles is the cationic nature of the solvent-exposed surface. The colloidal particles have a zeta potential of from about 1 mV to about 400 mV, preferably from about 20 mV to about 100 mV.

One concern for the pharmaceutical industry is the realization that many pipeline “investigational new drugs” are not readily soluble in water, thereby reducing bioavailability. This hurdle is often insurmountable and potentially effective drugs fail to clear clinical trials. The technology described here addresses this problem by packaging hydrophobic molecules in submicron colloids.

Advantageously, the colloidal particles can be prepared for targeting of specific cell surface receptors through adduction of the C-terminal lysines with different molecules or functional groups, such as cholesterol, mannose, TAT peptide, insulin, biotin, nucleotides, or any other suitable known surface targeting molecules, and combinations thereof. The colloidal particles having such targeting moieties conjugated to the exterior surface will therefore localize in and be selectively taken up by specific cells or tissues of a patient. Thus, the colloidal particles can be used for targeted therapies (gene therapy, cancer treatment, etc.), and nanodrug delivery by administering the colloidal particles having the targeting moieties to a patient. The targeting moiety is attached to the hydrophilic components of the peptides used to form the colloidal particles, which predominately occupy the outer layer of the particle, thus presenting the targeting moiety on the exterior surface of the colloidal particles after formation. The moiety will be recognized by the targeted region or tissue in the patient, and the colloidal particles will automatically localize in that region or tissue. Targeting these structures to specific cell types could reduce the amount of active ingredient required as well as limit off target effects.

The colloidal particles find application in the cellular deliver of poorly (water) soluble compounds/drugs that are currently too hydrophobic to be delivered effectively. As used herein, references to “poorly soluble” active agents refer to compounds and materials that have low solubility in aqueous solvent systems (e.g., having a solubility of less than 1 milligram per mL of the agent at neutral pH in a physiological buffer, 37+/−1° C.), and are contrasted with agents that can be fully dispersed or dissolved in aqueous systems. This allows small molecules with poor solubility and low cellular permeability to become viable drug candidates. There are numerous synthetic and plant oils that are able to preferentially able to solubilize hydrophobic molecules. The foregoing technology would be useful to prepare, for example, insecticides, fungicides, anti-parasiticides, anti-cancer drugs, and improving the bioavailability of many lipid-soluble active ingredients. In particular, the present technology is particularly suited as a delivery vehicle for formulating BCS Class II (low solubility, high permeability) and Class IV (low solubility, low permeability) drugs with poor solubility and poor bioavailability. The biopharmaceutics classification system (BCS) is a scientific approach based on the aqueous solubility and intestinal permeability characteristics of the drug substance or substances.

The colloidal particles can be used in pharmaceutically-acceptable compositions for delivering the colloidal particles to a subject. In one or more embodiments, the composition comprises a therapeutically-effective amount of colloidal particles dispersed in a pharmaceutically-acceptable carrier. As used herein, a “therapeutically effective” amount refers to the amount of the colloidal particles that will elicit the biological or medical response of a tissue, system, animal, or human that is being sought by a researcher or clinician, and in particular elicit some desired therapeutic effect. One of skill in the art recognizes that an amount may be considered therapeutically effective even if the condition is not totally eradicated but improved partially. As used herein, the term “pharmaceutically-acceptable” means not biologically or otherwise undesirable, in that it can be administered to a subject, cells, or tissue, without excessive toxicity, irritation, or allergic response, and does not cause any undesirable biological effects or interact in a deleterious manner with any of the other segments of the composition in which it is contained. A pharmaceutically-acceptable carrier would naturally be selected to minimize any degradation of the colloidal particles, functional groups, or active gents, and to minimize any adverse side effects in the subject, cells, or tissue, as would be well known to one of skill in the art. Pharmaceutically-acceptable ingredients include those acceptable for veterinary use as well as human pharmaceutical use. Exemplary carriers and excipients include aqueous solutions such as normal (n.) saline (˜0.9% NaCl), phosphate buffered saline (PBS), and/or sterile water (DAW), oil-in-water or water-in-oil emulsions, and the like.

Also described herein is a method of targeting delivery of an active agent to a region of a patient comprising administering to a patient, colloidal particles as described herein, which comprises a targeting moiety on the exterior surface. The moiety will be recognized by the targeted region or tissue in the patient, and the colloidal particles will automatically localize in that region or tissue. The colloidal particles can be injected directly into the target tissue, or can be administered systemically.

The colloidal particles are taken up by cells through the endocytic pathway where they are later metabolized in the cells to release their payload/content and/or any surface-conjugated materials. In particular, the cationic surface of the colloidal particles allows them to be taken up by the cell membrane which forms an early endosome. The colloidal particles begin to break down in the late endosome releasing their contents in the perinuclear cytosol. Further, the new pH of this intracellular environment results in a reduction of electrostatic attraction, and the surface payload, if any, is released. In this manner, surface bound nucleic acids and any encapsulated active agents are released from the colloidal particles into the cytosol.

The colloidal particles have been shown to effectively deliver nucleic acids, which are released in a time-dependent manner. The colloidal particles can also be used for surface binding of proteins, peptides, plasmids, and nucleic acids, including CRISPR-Cas9 components for delivery. And they can be used to encapsulate a wide variety of active agents, including small molecules, fat soluble vitamins, pheromones, and fatty acids.

Also described herein are methods of delivering active agents to plants, such as to the leaves, stems, roots, or other tissues or cells of the plant. The methods can be used to deliver a variety of active agents, including to treat and/or prevent pests, disease, infection, and the like. The method comprises applying the colloidal particles to at least a portion of a plant and/or to the soil where a plant is or will be planted.

Thus, also contemplated herein are methods for delivering active agents to a plant, animal, or human. The methods comprise administering a plurality of colloids containing an active ingredient to the plant, animal, or human. This can include directly applying or administering the colloids, or providing the colloids to the vicinity of the target. For example, the colloids may be applied directly to a plant leaf or root system, or may be applied to the soil around the roots. Likewise, the colloids can be directly administered topically, orally, or via injection into the animal, or may be introduced indirectly, for example, into aquaculture/water system in which the animal resides, or in a location where the animal may come into contact with it (e.g., near a beehive, etc.). The colloids may be incorporated into a suitable pharmaceutical, horticultural, or veterinary composition, including a suitable carrier, diluent, excipient, or vehicle for administration.

Also described herein are methods of delivering active agents to insects by contacting an insect with colloidal particles carrying the active agent, such as an insecticide. The methods can comprise applying the colloidal particles to the leaves, stems, roots, or other tissues or cells of the plant, or otherwise placing the colloidal particles in a location where the insects/pests will come into contact with the colloidal particles. In some embodiments, the colloidal particles may be ingested by the insects. In some embodiments, the colloidal particles can be provided in an insect bait, along with an edible insect attractant (sugars, carbohydrates, yeast, fats, oils, proteins). The bait can be in the form of a liquid, gel, or solid tablet or granules.

The technology described herein can be used to deliver a wide variety of active agents, including, without limitation, imaging agents, detectable dyes, fungicides, anticancer agents, insecticides, herbicides, metabolic inhibitors, etc.

In some embodiments, the peptides, colloids, or compositions can be provided in unit dosage form in a suitable container. The term “unit dosage form” refers to a physically discrete unit suitable as a unitary dosage for human, plant, or animal use. Each unit dosage form may contain a predetermined amount of peptides, colloids, or compositions in a suitable carrier calculated to produce a desired effect. In one or more embodiments, kit comprises lyophilized colloids, wherein the pre-formed colloids have been freeze-dried or spray-dried, along with instructions for reconstituting the lyophilized colloids for use. In one or more embodiments, kit comprises the peptides, colloids, or compositions, with instructions for preparing the colloids or composition and administering the composition to the subject.

It will be appreciated that therapeutic and prophylactic methods described herein are applicable to humans as well as any suitable animal, including, without limitation, dogs, cats, and other pets, as well as, rodents, primates, horses, cattle, pigs, etc. The methods can be also applied for clinical research and/or study. This platform technology is also useful in plants, such as for targeting pathogens in plant system or otherwise delivering various active agents to plant tissues or their pests or pathogens.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Example 1

### Linear Amphipathic Oligopeptides Stably Encase Solutes Dissolved in Low Dielectric Oils/Solvents Dispersed in Water

### Abstract

This report introduces amphipathic peptides that stably encapsulate oils and low dielectric solvent droplets in water. The amphipathic peptide corrals these liquids yielding monodispersed ˜20-2000 nm colloidal particles that can be resized. They are stable for long periods of time and over a temperature range of 4-90° C. The cationic colloids possess Zeta potentials ranging from −6.1 to +50 mV. The peptides remain unstructured with the lysyl-residues fully solvent exposed. Encapsulated coconut oil retains its principal phase transition indicating that the oil remains in the liquid state. Lastly these capsules are rapidly taken up by cells in culture suggesting that these oil-filled colloids could potentially find application in delivering hydrophobic therapeutics.

### Materials and Methods

Peptide synthesis—The test peptides based on the Ac-FLIVI-KKKKK-CO-NH₂ (SEQ ID NO: 6) sequence were synthesized using standard synthesis. The amino acids (F, I, L, V, A) along with HOAt and HATU were obtained from either P3 Biosystems (Loiusville, KY) and (K) from AnaSpec Inc. (Fremiont CA). DMF was from (Thermo-Fisher, Waltham, MA), N-Methyl-2-pyrrolidone (Sigma-Aldrich Corp. St. Louis, MO), Piperidine (American BioAnalytical, Canton, MA) and CLEAR® Amide resin (Peptides International, Louisville, KY)

Peptides were cleaved in 1 M HCl (Thermo-Fisher, Waltham, MA) in 1,1,1,3,3,3-Hexafluoroisopropyl alcohol (HFIPA) (Sigma-Aldrich Corp. ST Louis, MO) for 4 h at RT. After cleavage, the peptides were dried in vacuo. The calculated mass of the peptide was confirmed by mass spectrometry. The 5 (6)-carboxyfluorescein (5 (6)-CF) labeled peptide was prepared by deprotecting the bound Boc-protected lysyl-epsilon-amino groups with 20% TFA in dichloromethane (DCM) for 20 min. After washing with the deprotected peptide with DCM, 5 (6)-carboxyfluorescein was added (at a concentration of 1 part to five parts lysine) in the presence of HOAT-HATU in DMF for 20 min. The reaction was stopped by filtering the dye and then washing with more DCM. This material was cleaved from the resin and analyzed as described above. MALDI-mass spectral analyses showed that the products of this synthesis with the partial coupling of CF were the unlabeled peptide and the mono-substituted one.

Encapsulation studies-Winterized Soybean oil (Archer Daniel Midlands, Chicago, IL) 50 μL was added to 2 mg of dry peptide and vortexed for 2 min. Deionized distilled-reverse osmosis (DDI-RO) water was added to a final volume of 1 mL. This mixture was sonicated (bath sonicator) for 15 min at 37° C. The generated CAPC are maintained at RT for at least 1 h prior to confocal microscopy. For the solvent/oil encapsulation study reagent grade-Benzene (Sigma-Aldrich, St. Louis, MO), Cyclohexane (Acros Organics, Geel, Belgium), n-Decane (grade 99+%, Sigma-Aldrich, St. Louis), Migloyl® 812N; capric triglyceride (Pharma Grade, IOI Oleo GmbH, Witten, Germany), and Mineral oil paraffin (USP, Thermo-Fisher Scientific, Waltham, MA) and Winterized Soy oil (Archer Daniel Midlands, Chicago, IL) were used. These CAPCs were prepared using 50 μL of the solvent/oil along with 480 μL of DDI-RO water were sonicated for 15 min. CAPC, 2 mg, was subsequently added and sonicated for 10 min. These samples were measured 24 h thereafter for their size, Zeta potential and their circular dichroism (CD) spectra recorded.

Particle size/Zeta potential—The samples were analyzed using a Litesizer™ 500 Particle Size Analyzer (Anton Paar GmbH, Graz, Austria) using an Omega Cuvette at 25° C. The size distribution of the particles was analyzed with the proprietary Kalliope™ v.2.16 software (Anton Paar GmbH, Graz, Austria) using the intensity-weighing model. For the Zeta potential analysis, the suspension used above was diluted to 5-fold in DDI-RO prior to analysis.

NTA studies—NTA measurements were performed with a NanoSight LM 14 (Malvern Panalytical), using a sample chamber connected to a 405 nm laser and equipped with Hamamatsu Photonics K. K. CMOS camera Model #C11440-50B. CAPCs samples were injected in the chamber with sterile syringes (BD Discardit II, New Jersey, USA) and particles tracking performed with 5 individual captures of 25 frames/second in a time lapse of 60 seconds. All measurements were performed at 25° C. and captured images analyzed by NanoSight NTA 3.3 software to calculate the concentration and size of the nanoparticles in suspension.

Circular Dichroism—The same samples used for Zeta potential measurements 50-100 μM CAPC solutions were analyzed using a Jasco J-815 CD spectrophotometer (Jasco Analytical Instruments, Easton, MD). The samples were scanned from 260-190 nm at 50 nm/min with 1 nm step intervals in a 1 mm path-length rectangular cuvette.

Differential Scanning calorimetry-Differential scanning calorimetry (DSC) experiments were performed with a VP-DSC calorimeter (MicroCal Inc., Northampton, MA) at a scan rate of 1K/min. The instrument baseline was obtained first by measuring the heat-capacity profile of the water blank and each measuring subtracted from the reference baseline.

Temperature Stability-Temperature stability was screened with CAPC composed of 3 mM peptide containing coconut oil. The particles in suspension were diluted to 2% in DDI-RO for particle size analysis and to 0.05% for determining Zeta potential. Samples were analyzed using a Litesizer™ 500 Particle Size Analyzer (Anton Paar GmbH, Graz, Austria) using an Omega cuvette over a 5 to 70° C. range.

Shelf-life stability—A CAPC sample containing a lipid mixture was prepared as previously described and characterized for average size, surface charge and polydispersity. A fraction of this sample was set aside for stability studies at room temperature in the dark. At random intervals the stored sample was retested for these three parameters.

Confocal Microscopy—CAPCS were prepared with both CF labeled peptide and soy oil containing Nile red (TCI America, Portland, OR). This dye is insoluble in water and only fluoresces in lipids and organic solvents. It has previously been used to allow visualization of encapsulated lipids using confocal microscopy. The two, dye containing CAPC, suspended in water, were combined with low melting agarose 0.7% in a ratio of 1:1 to immobilize the particles. A 20 μL drop was spotted on a microscope slide with a cover slip applied shortly thereafter. Single confocal sections were taken using the 20× air and 63× oil objectives. The 488 nm filter allowed visualization of the CF peptide and the 594 nm filter was used to image the Nile Red. The preparations were imaged using a Zeiss LSM 700 (Zeiss Inc., Carl-Zeiss-Strasse 22, 73447 Oberkochen, Germany) processed using the Zeiss Zen software images that were exported as jpeg files for publication.

Resizing of CAPC—Samples averaging >600 nm diameters prepared as described in the previous section were individually loaded in gastight syringes and extruded through a 0.2 micron pore 19 mm Whatman Nuclepore Track-Etched polycarbonate extrusion membranes Avanti Polar Lipids, Inc., Alabaster, AL); and additionally for some experiments through a 0.1 micron pore 19 mm Whatman Nuclepore Track-Etched polycarbonate extrusion membranes passing through the Avanti Mini-Extruder (with at least 25 passes through the membrane per sample. The two-step process was necessary as attempts to resize directly through the 0.1 micron were unsuccessful. Resized samples were used in the cell uptake experiments.

TEM studies—Transmission electron microscopy (TEM) images were produced using a Hitachi STEM 4800 EM (Hitachi High Tech Group Europark Fichtenhain A12, 47807 Krefeld, Germany) with samples being prepared and resized to 0.2 micron as previously described. The peptides used for this experiment were labeled with Me-Hg. A droplet of each sample (20 μL) was placed onto a 200-mesh Formvar-coated grid (Lacey TEM grids for 10 min, and the excess sample was wicked off using filter paper. Samples were given 30 min to dry in open glass petri dish set into drying oven at 55° C. prior to visualization.

Cell Uptake Studies-CHO cells (ATCC, 10801 University Blvd. Manassas, VA) were grown in Dulbecco's Modified Eagle Medium/Ham's F12 (DMEM/Ham's F12) (Gibco, USA) supplemented with 10% (v/v) fetal bovine serum (FBS) (Gibco, USA), 1% Penicillin/Streptomycin (Gibco, USA) and maintained in a humidified incubator at 37° C. with 5% CO₂. For the cellular uptake experiment, cells were seeded at 2.5×10⁴ Cells per well in a Thermo Scientific Lab-Tek II CC2, 8 well glass microscope slide chamber (Thermo Fisher Scientific, Waltham, MA, USA) and kept in growth media to reach a confluence of 60-70%. Prior to adding the CAPCs, growth media was replaced by Opti-MEM Reduced Serum (Thermo Fisher Scientific) and CAPCs with caboxyfluorescein labeled peptide and Nile Red dissolved in the oil were mixed to medium to a final concentration of 5% in 200 μL final volume. These CAPCs were resized as described above and incubated with cells for 4-6 h at 37° C. and 5% CO₂ and fixed with 4% Paraformaldehyde followed by incubation with 0.2% Tween 20 for cell permeabilization and slide mounted using ProLong Gold Antifade Mountant with DAPI (Thermo Fisher Scientific) for nucleus staining and images obtained by confocal microscopy as previously described.

### Results and Discussion

### Topography Study

The positive Zeta potential for the CAPCs Table 1 indicate that the peptides are orienting with their oligo-lysyl ε-amino groups solvent exposed.

TABLE 1
Properties of CAPC encapsulating different
of oils/solvents dispersed in water

| | Diameter | Polydispersity | Zeta Potential |
|---|---|---|---|
| Hydrophobic Core | (nm) | (%) | (mV) |
| Cyclohexane | 220/389 | 30 | 15.9 |
| Benzene | 168 | 27 | 16.6 |
| n-Decane | 804 | 27 | 49.8 |
| Mineral oil paraffin | 687 | 36 | 26.8 |
| Soy oil | 465 | 29 | 44.8 |
| Migloyl ® 812N* | 469 | 5 | 22.1 |
| Coconut Oil | 531 | 26 | 24.2 |

*Lipid component in parenteral nutrition. Neutral, stable, solubilizer and carrier for oil soluble actives containing a triglyceride ester of saturated coconut/palm kernel oil derived caprylic and capric fatty acids and plant derived glycerol.

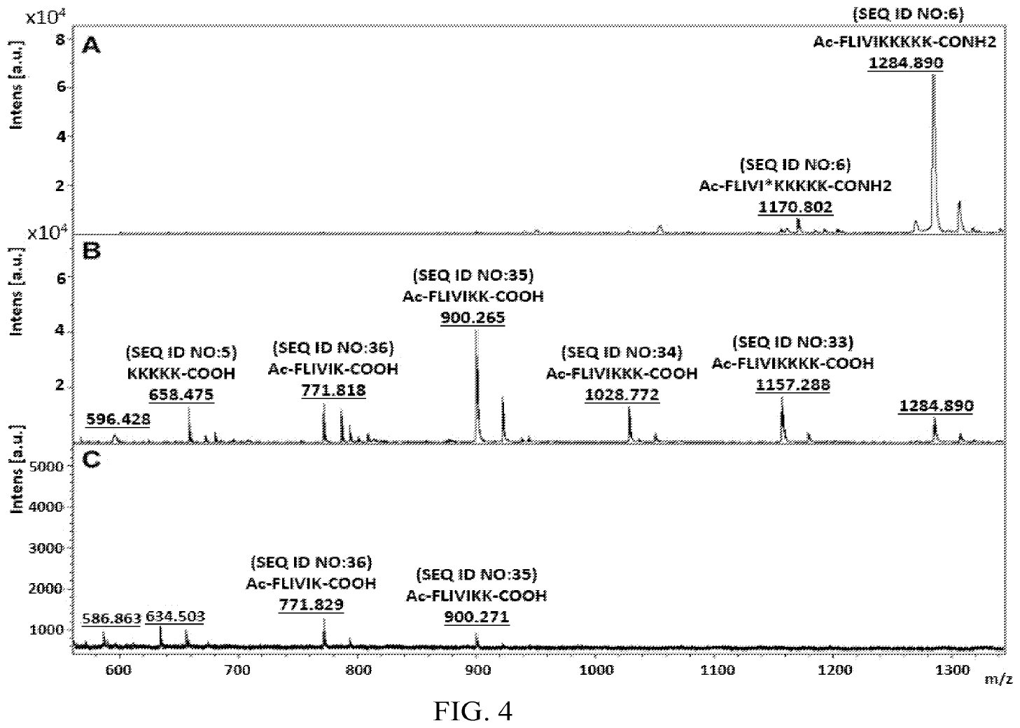

This orientation of the lysyl-residues was confirmed by performing trypsin digests on preformed CAPCS, and on the peptide aggregates formed in water serving as a control (FIG. 4). In FIG. 4 (S1-A), the mass spectrum of intact CAPC peptide displays the expected mass of 1284.89 Da. By 1 hr (S1-B) segments were observed with one to four lysyl-groups remaining. The 1 h control (S1-C, aggregated peptide) was nearly fully digested. This result suggests there may be degrees of solvent accessibility for the oligo-lysines in CAPC. Lysyl-residues can become partially buried in hydrophobic environments with their epsilon amino groups snorkeling outward to reach the aqueous phase, particularly in membranes.

### Biophysical Studies:

The first experiments documenting the complexation of the initial CAPC peptide sequence Ac-FLIVIKKKKK-CO-NH₂ (SEQ ID NO:6) with soy oil droplets focused on DLS, MALDI-MS and CD measurements. The samples were analyzed for their hydrodynamic size and polydispersity by dynamic light scattering, as well as their zeta potential. The diameters of the particles measured were derived from an assessment of the DLS intensity peak. After the sonication step the mixture was cloudy with no free oil coalescing on the surface of the sample. The average (mean) diameter was 465 nm with a positive Zeta potential of 44.8 mV (Table 1). These samples were subjected to trypsin digestion to assess the aqueous accessibility of the Lysyl-residues (FIG. 4). Within 1 h, all 5 of the lysine residues were proteolytically released indicating that the protease had access the carboxyl-end of each residue. The circular dichoism spectrum for this mixture assessed the conformational state of the Ac-FLIVIKKKKK-CO-NH₂ (SEQ ID NO:6) peptide in CAPCS. The CD spectra had a strong minimum at 198 nm with a slight maximum at ˜218 nm. This spectrum is consistent for peptides in a random coil conformation.

This initial study was performed using a natural mixture of triacylphospholipids containing primarily 18:1 and 18:2 phospholipids. Subsequently we tested a variety of oils and non-polar solvents for encapsulation-following their diameters, polydispersity index and Zeta potential (Table 1).

All of the tested solvents and oils were encapsulated by the peptide producing capsules at 37° C. with positive Zeta potentials. With the exception of Cyclohexane, the others yielded single average diameter populations. With the exception of Mineral oil (>30%), they had good poly-dispersion indices. The average diameters of the capsules were quite variable with a low of 168 nm for Benzene and a high of 804 nm for n-Decane. Most of the oils, excluding Mineral oil, are straight saturated or bent unsaturated alkyl-chains and yielded colloid diameters in the 450-530 nm range. Mineral oil contains bulkier unsaturated branched or polycyclic alkyl chains that produce almost 700 nm diameter colloids. The larger diameter of the n-Decane colloids does not appear to fit a model that would predict that the molal volume of the hydrophobic oil or solute dictates the size of the peptide encapsulated colloid.

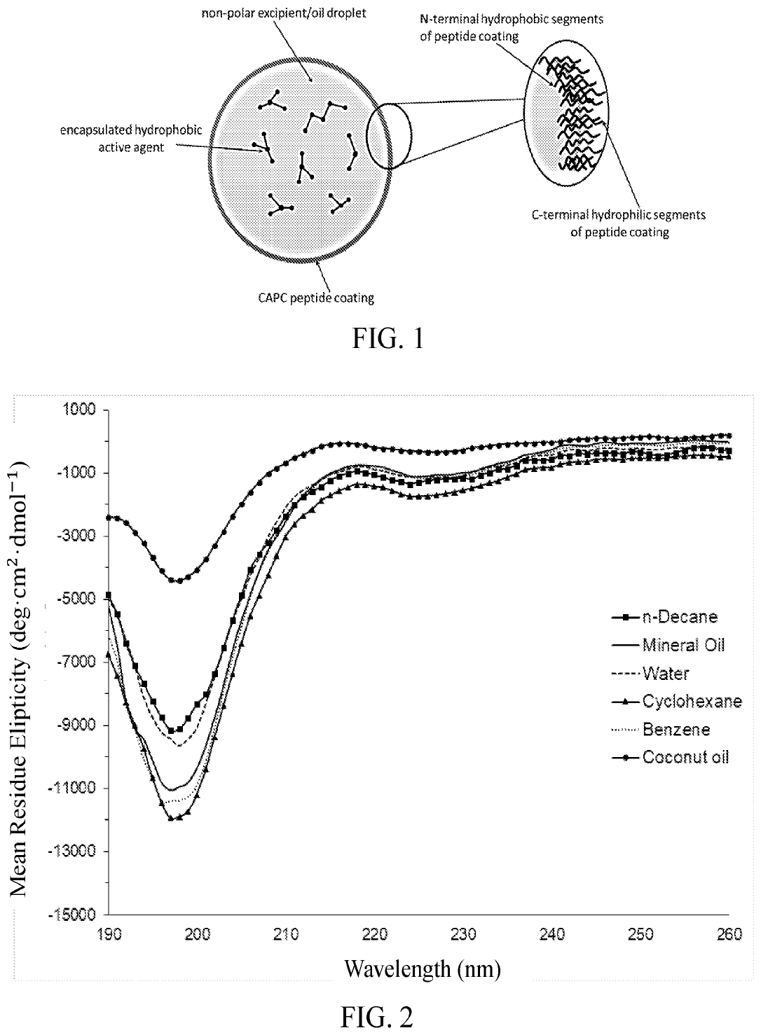

Circular dichroism was employed to determine the secondary structure of the encapsulating peptides for each of the Table 1 solvents (FIG. 2). The CD spectra for all of the colloids look remarkably similar, all having strong minimum at 198 nm with a slight maximum at ˜218 nm. These spectra are consistent with peptides in a random coil conformation. The absence of beta-structure implies the assembled peptides are not interacting through hydrogen bonds with each other but rather through peptide-peptide hydrophobic contacts and the oils/solvents.

Six different peptide sequences were prepared to test if any short hydrophobic sequence in the amphipathic peptide would effectively encapsulate soy oil (Table 2). For all sequences, a phenylalanine residue is present, allowing for concentration determinations. One sequence includes a C-terminal cysteine to facilitate the attachment methyl mercury (Me-Hg) that was used in our TEM study. Secondary structural analyses and size and charge determinations were performed.

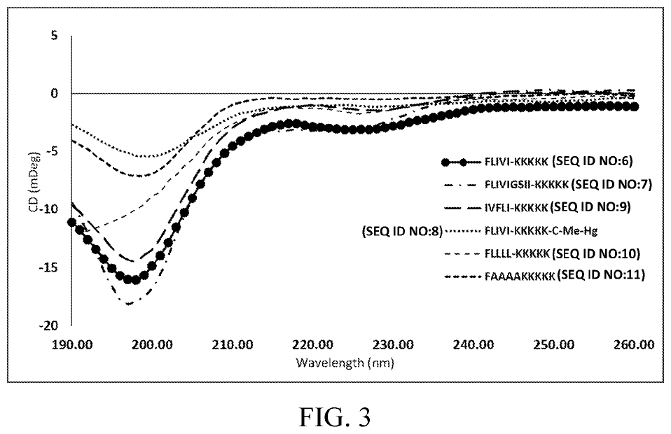

All of the non-polar sequences were able to form colloids. Yet not all yielded useful sizes and degrees of polydispersity. Of note is the observation that all of the peptides interacting with the soy oil remained unstructured based on their CD spectra (FIG. 3). Branched versions of similar peptide sequences with two lysine tails are strong beta-forming sequences capable of forming self-assembling peptide bilayers in water. As linear sequences immersed in oil, they do not adopt beta-structures.

TABLE 2
Colloids produced by different amphipathic sequences in soy oil. The calculated AG is
for moving the bolded hydrophobic segment from water to octanol and serve as a reference for
relative hydrophobicity.

| | Hydrodynamic | | Zeta Potential |
|---|---|---|---|
| Peptide Sequence | Diameter (nm) | Polydispersity (%) | (mV) |
| Ac-FLIVI-KKKKK- | 560 | 20 | 38 |
| CONH₂(SEQ ID NO: 6) | | | |
| 32.7 ΔG (kcal/mol) | | | |
| Ac-FLIVIGSII- | 300 | 23 | 21 |
| KKKKKCONH₂(SEQ ID NO: 7) | | | |
| 42.3 ΔG (kcal/mol) | | | |
| Ac-FLIVI-KKKKK-C-Me- | 578 ± 96.0 | 25 ± 5.8 | 33 ± 4.0 |
| Hg(SEQ ID NO: 8) | | | |
| 32.7 ΔG (kcal/mol) | | | |
| Ac-IVFLI-KKKKK- | 594 | 25 | 25 |
| CONH₂(SEQ ID NO: 9) | | | |
| 32.7 ΔG (kcal/mol) | | | |
| Ac-FLLLL-KKKKK- | 666 | 200 | −0.6 |
| CONH₂(SEQ ID NO: 10) | | | |
| 37.4 ΔG (kcal/mol) | | | |
| Ac-FAAAA-KKKKK- | 681 | 500 | 10.2 |
| CONH₂(SEQ ID NO: 11) | | | |
| 6.8 ΔG (kcal/mol) | | | |
| 5(6)-Ac-FLIVI-KKKKK- C- | 766 ± 225.0 | 21 ± 4.8 | 25 ± 2.7 |
| CF(SEQ ID NO: 8) | | | |
| 32.7 ΔG (kcal/mol) | | | |

Examining their sizes and charges, they form CAPC with average diameters of 560 and 300 nm, respectively. Compared to the Ac-FLIVI-KKKKK-CONH₂ (SEQ ID NO:6) soy oil CAPC shown in Table 1 the average diameter in Table 2 is slightly larger. We do see batch to batch differences in diameters that range from 400-700 nm. The larger sequence yields a smaller CAPC with a lower Zeta potential. The methyl mercury containing sequence behaved similarly to the parent sequence containing the FLIVI (SEQ ID NO: 1) segment. This is expected as the adducted mercury occurs on the part of the peptide that is aqueous exposed. The scrambled sequence containing the sequence IVFLI (SEQ ID NO:27) produced CAPCs that were quite similar to the parent sequence indicating that the order of the amino acids is not important. The two outliers in this study are the sequences containing repeating leucine or alanine residues. The oligo-leucine containing sequence has a similar AG as the parent sequence. Isoleucine and valine are structurally different from leucine in that they both possess internal branched methyl groups on the beta carbon. The oligo-alanine peptide, while still non-polar, is considerably less hydrophobic, reducing its ability to interact with the hydrophobic droplets. Both display considerably higher polydispersity values and lower Zeta potentials. Both of these parameters indicate a propensity for aggregation. Together these values suggest these two sequences are actually forming smaller CAPCs that are subsequently aggregating to yield the diameter values reported in Table 2. The fact that the parent sequence contains beta-substituted amino acids (Ile and Val) may have some effect on CAPC properties. The smaller CAPC formed by the longer sequence (Ac-FLIVIGSII-KKKKK-CONH₂ (SEQ ID NO:7)) was not expected. It appears that the addition of the four extra amino acids may limit the volume in the oil droplets that form during the sonication step. Due to the additional cost of producing the longer sequence and the fact that the shorter CAPCs and be resized (see below) this sequence was not tested further.

During initial CAPC formation various sizes are formed ranging from nm to micron sizes (shown later in the microscopy section). Their flexibility allows them to be resized by extrusion sequentially thorough a 200 nm pore and then 100 nm pore a polycarbonate filter (Table 3). Given that cationic 100 nm nano-spheres are readily taken up by cells freshly prepared CAPC were sequentially extruding 21-times thorough a 200 nm and then 21-times through a 100 nm pore polycarbonate membrane filter (Table 3). Attempts to pass the initial heterogeneous suspension through the 100 nm filter were unsuccessful due to high resistivity.

TABLE 3
Lipid control and resizing of soy oil encapsulated CAPC

| | N value | Pd | ZP |
|---|---|---|---|
| SAMPLE | (nM) | (%) | (mV) |
| Soy oil (lipid control) | 216.7 | 39.4 | −42.9 |
| CAPC prior resizing | 318.8 | 28.0 | 22 |
| CAPC after extrusion (200 nm pores) | 198 | 23.6 | 21 |
| CAPC after 2^{nd} extrusion (100 nm pores) | 95.7 | 22.4 | 10.5 |
| CF-CAPC after extrusion (200 nm pores) | 155 | 21.0 | 31 |
| Extruded CAPC prior drying (200 nm pores) | 220 | 19.3 | 18 |
| Extruded CAPC after rehydrating | 160 | 21.0 | 22 |

The hydrodynamic value of the CAPC pre-resizing shows the typical heterogeneous population of sizes with good polydispersity and a positive zeta potential. After repeatedly extruding the heterogeneous mixture back and forth solution through the 200 nm pore filter a lower hydrodynamic diameter value is obtained with retention of similar polydispersity and zeta potential values. After the second extrusion across the 100 nm pore the average hydrodynamic diameter value is reduced further. The polydispersity is unchanged however the zeta potential while remaining positive has a decreased value.

These results can be interpreted from a surface area perspective. When CAPC form, a finite concentration of cationic residues (lysines) is distributed on the colloid surface. As the CAPC are extruded through the filter the total number of CAPC suspended increase with a concomitant increase in total surface area of suspended particles. As the summed surface area of the sample increases the number of peptides on the surface of each CAPC must necessarily decrease. The similarity between the starting CAPC preparation and the 200 nm extruded sample suggest that the bulk of the starting sample have similar surface areas to those of the extruded sample. Acknowledging that a range of sizes are initially formed, the distribution must be asymmetric and skewed toward smaller CAPC, roughly 200 nm. When the sample is resized for the second time through the 100 nm filter with the summed surface area increasing, the zeta potential now shows the anticipated drop in the zeta potential.

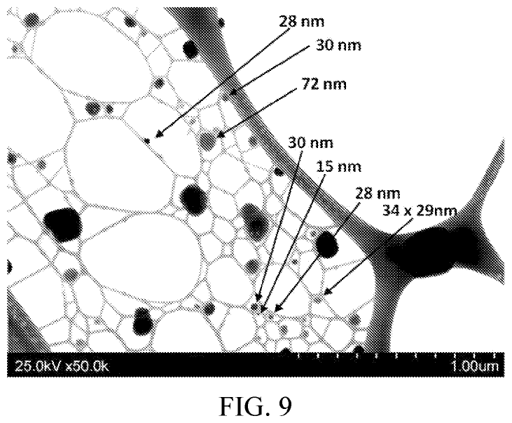

This table also includes data on resized CAPC that were lyophilized and subsequently rehydrated. The rehydrated CAPC were slightly smaller however their polydispersion index and Zeta potential were unchanged. Confocal microscopy of these samples are shown in the microscopy section (FIG. 9). The ability to generate dried CAPC allows for the preparation of more concentrated samples upon rehydration with reduced water volumes.

TABLE 4
Measured DLS parameters of CAPC colloids before resizing after
resizing and again after being freeze dried and rehydrated.

| SAMPLE | SIZE (nM) | PD (%) | ZP (mV) |
|---|---|---|---|
| CAPC prior to resizing | 761 ± 211.0 | 28.0 ± 6.5 | 22 ± 11.0 |
| Extruded CAPC prior drying (200 | 220 ± 20.0 | 19.3 ± 2.3 | 18 ± 2.5 |
| nm pores) | | | |
| Extruded CAPC after rehydrating | 160 ± 42.0 | 21.0 ± 5.0 | 22 ± 4.2 |

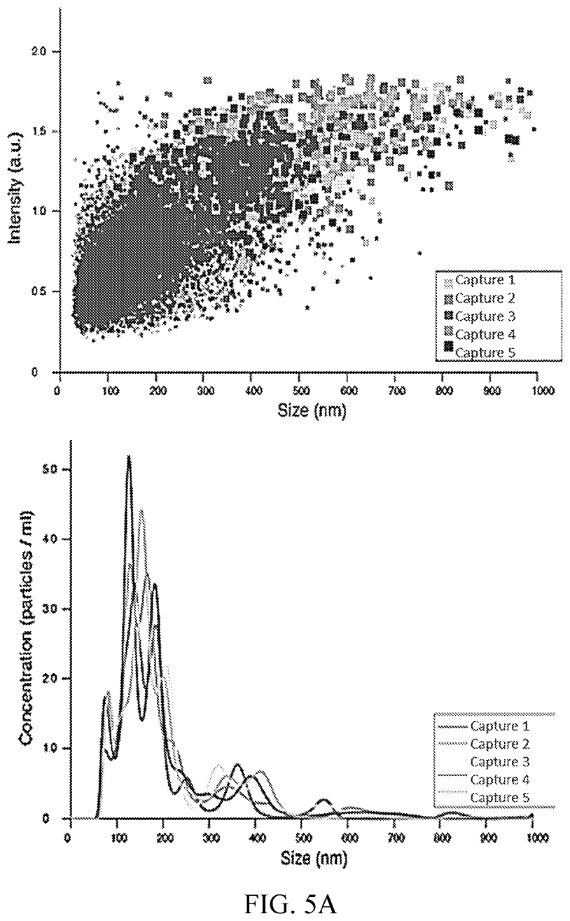

Using DLS, while helpful as a first step, does not provide size distribution and concentration information. Nanoparticle Tracking Analysis (NTA) was employed to study the distribution of the CAPCs just after formation (FIG. 5A) and again when resized (FIG. 5B). This technique uses direct observation diffusion events to produce high resolution nanoparticle size distribution and particle concentration data information.

The initial preparation yielded 2.16×10⁸+/−1.83×10⁷ particles/mL particles with a mean of 188.2 nm, a standard deviation of 142.2 nm with 90% of the particles <349.7 nm. After resizing through the 200 nm filter there were 1.07×10⁹+/−2.72×10⁷ particles/mL particles with a mean of 146.1 nm, a standard deviation of 75.5 nm with 90% of the particles <247.2 nm.

This data, compared to the DLS analyses, gives a better representation of the size distribution of the preparation before and after resizing. Upon resizing note that the number of particles increases by about 5-fold as the larger particles are resized into small ones.

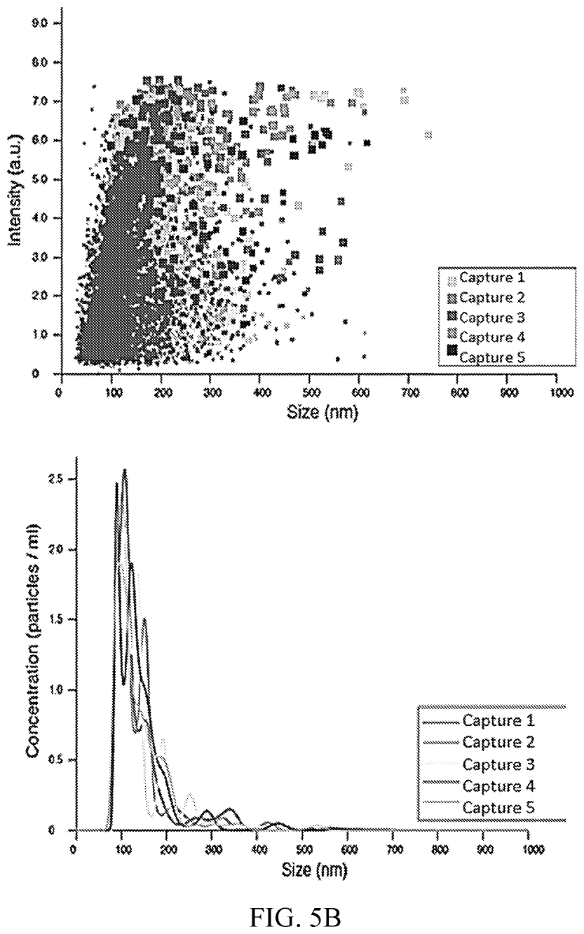

A thermal stability study was conducted where we replaced the soy oil with coconut oil which has a phase transition around room temperature. This tested whether the liquid/solid phases of the oil influenced the stability of the coconut oil/peptide colloid. After preparing the CAPC at 37° C. they we either cooled or heated in our temperature-controlled particle size analyzer. They were held at the new temperature for 5 min prior to taking the reading. For this experiment 200 nm sized CAPCs were tested (FIG. 6). At all temperatures CAPC remained intact and the sizes of CAPC maintained at or above the phase transition temperature for coconut oil had similar diameters. Below the phase transition temperature, the diameters decreased by roughly 7% and 10% for the 15° C. and 5 & 10° C. samples respectively. As the lipid transitions to the solid-state motion of the side chains is reduced allowing for more uniform packing. A molecular dynamics simulation studying the phase transition of hydrated DPPC and DPPE bilayers showed that below the phase transition temperature the lipids become ordered and have reduced molal volumes. While not shown, the sizes of the colloids are reversible for the low and high temperature CAPC when returned to 30° C.

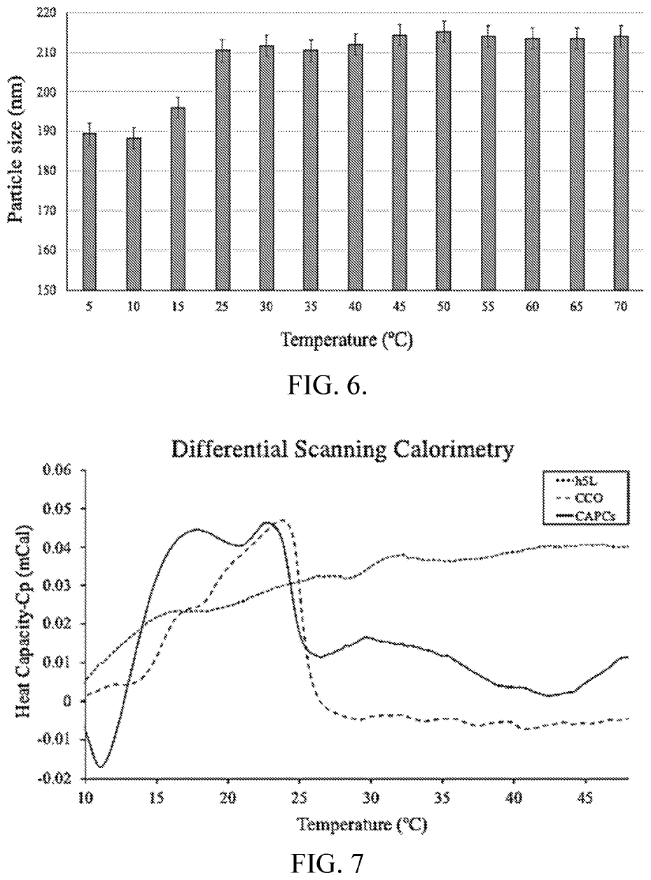

For the next studies we again prepared the colloid with coconut oil. Coconut oil has a phase transition around 24-25° C. that can be followed using differential scanning calorimetry. This property allowed us to evaluate CAPC stability when their contents are in the solid or liquid phase. In FIG. 7 we compared the phase transition of sonicated coconut oil droplets in water compared to peptide alone and encased in Ac-FLIVIKKKKK-CONH (SEQ ID NO:6) CAPC. The peptide by itself showed no phase transition over the temperature range used. The sonicated oil droplets exhibit the expected major phase transition between 24-25° C. The CAPC show three transitions with a minor broad one between 17-20° C. and a second major one around 23-24° C. and a very broad one between 30-42° C. This data suggests that the bulk of the encapsulated coconut oil is unbound transitions from solid to liquid normally. The 17-20° C. transition suggests a fraction of the oil interacts weakly with the peptides such that it lowers their melting temperature (Tm). The 30-42° C. fraction transition could indicate a gel-like state with a fraction of the lipid that dissociates at elevated temperatures. At normal body temperatures nearly all of the coconut oil is in the liquid state.

In a recent paper exploring mixtures of short aromatic hydrophobic peptides with gasoline, kerosine or diesel fuels, temperature reversible gels formed that were stabilized through Pi-Pi stacking and hydrophobic interactions. The inclusion of the phenylalanine in each of the sequences could be facilitating the self-assembly and stability of the peptides in CAPC.

### Stability Study-

A CAPC preparation was prepared to analyze long term stability (Table 5). The test sample contained 100% of a mixture of two proprietary liquid fatty acid pheromones. Throughout the course of the experiment the sample was monitored with a particle size/Zeta potential analyzer. Over the entire time course, particle sizes, poly dispersity and Zeta potential remained remarkably consistent. These results speak to the long-term stability of these particles and the fact that they do not undergo fusion into larger particles. The current results indicate that CAPC are a robust colloid. Since no reactive active ingredient (AI) was present there is no data on the ability CAPC to protect against photo, redox or other types of inactivation. AI long-term stability experiments are currently underway. Freeze-dried and spray-dried material stored in the dark will hopefully have even longer half-lives as well as offer UV protection for light sensitive active ingredients.

TABLE 5
Shelf-life stability of lipid filled CAPC kept at RT and
in the dark. As determined using hydrodynamic diameter,
polydispersity index (Pd) and Zeta potential (ZP).

| | Hydrodynamic diameter | | |
|---|---|---|---|
| Measurement dates | (nm) | Pd (%) | ZP (mV) |
| Day 0 | 650 ± 105 | 24.4 ± 4.2 | 24.8 ± 4.0 |
| Day 15 | 644 ± 107 | 18.8 ± 2.4 | 30.2 ± 2.0 |
| Day 22 | 660 ± 112 | 21.7 ± 3.5 | 26.1 ± 4.0 |
| Day 29 | 695 ± 120 | 15.3 ± 9.0 | 21.9 ± 3.2 |
| Day 36 | 688 ± 118 | 10.4 ± 7.5 | 18.8 ± 6.5 |
| Day 57 | 644 ± 112 | 10.6 ± 8.2 | 24.6 ± 2.8 |
| Day 168 | 623 ± 123 | 19.9 ± 5.0 | 28.1 ± 5.0 |
| Day 408 | 640 ± 120 | 24.4 ± 4.2 | 23.4 ± 3.8 |

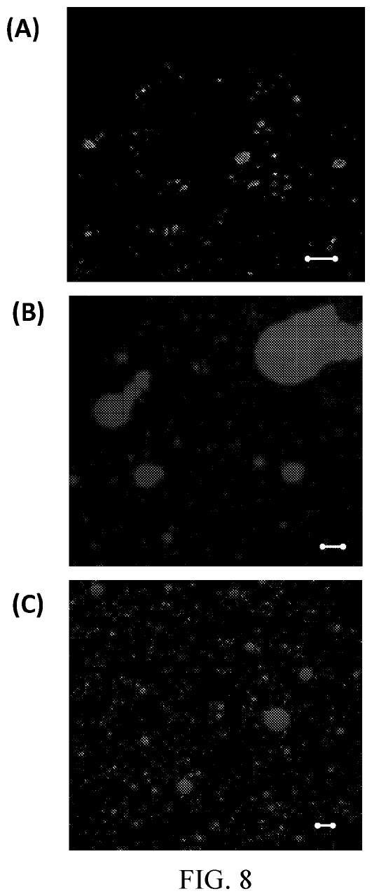

Microscopy Studies-Confocal microscopy images were generated in water with 1) sonicated CF-labeled CAPC peptide in water (left), 2) sonicated soy oil/Nile red (center) and 3) CF-labeled peptide sonicated in the presence of soy oil/Nile red (right) (FIG. 8). In FIG. 8 (left) the peptide in water (by itself) forms irregular spheroidal aggregates of different sizes, some approaching 10 nm in size. Some appear solid while others appear to have water-filled cavities. The soy oil/Nile red mixture right after sonication in water forms larger irregular oil blobs, 10 s to 100 μm in size. When peptide is mixed with the Nile red containing soy oil/peptide mixture and then sonicated in water well-ordered polydispersed capsules are formed ranging from around 3 microns down below 1 micron. Upon analyzing for the Nile red fluorescence and then superimposing the CF peptide signal (FIG. 8, bottom) the soy oil contents are clearly encased within the CAPC. The peptide coating, although quite thin, is visible as a green border surrounding the Nile red dye-colored oil. Notably, Nile red dye only fluoresces in hydrophobic environments and provides a good test case for hydrophobic active ingredients and therapeutic drugs.

Due to the size detection limits of confocal microscopy, we prepared CAPC using our Methyl-mercury labeled peptide to produce electron-dense CAPC that are detectable by TEM (FIG. 9). As shown in Table 2 labeling the peptide on the C-terminal aqueous exposed lysine residue does not affect the size, polydispersity or Zeta potential of the CAPC. Resized CAPCs were used, and the field chosen for this image focused on the smaller particle sizes. The diameters for some of the CAPC in this image range from 70 down to 15 nm. The larger ones are around 200 nm.

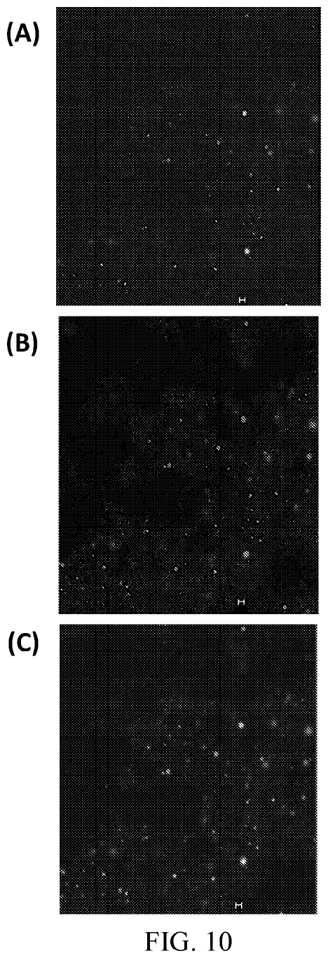

As part of our stability study, we subjected CAPC colloidal particles containing soy oil to freeze drying followed by rehydration (Table 3). The DLS studies indicated that rehydration of freeze-dried samples was possible (see also Table 4). Confocal microscopy was employed to verify if the encapsulated oil was still present after returning the particles to water (FIG. 10). The colocalization of the oil within the rehydrated CF-labeled CAPC shows that these robust particles that are not easily broken upon dehydration.

### Cell Uptake Study-

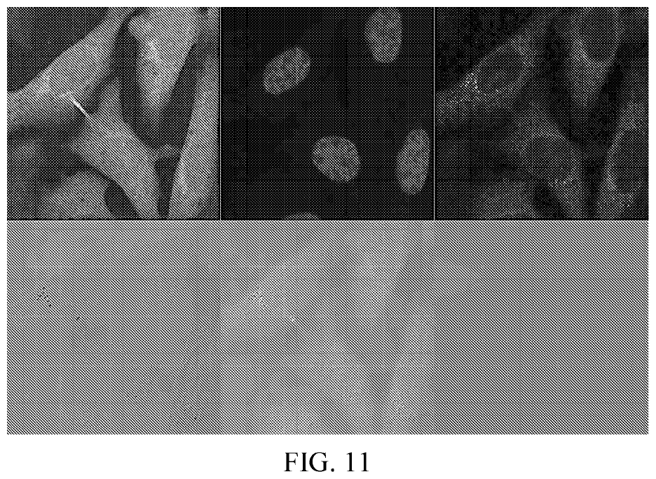

In designing the CAPC peptide we wanted to maintain the cationic surface of the assembled particles identical to our peptide bilayer delimited branched amphipathic capsules (BAPC). These water filled capsules are readily taken up by cells in culture and in vivo. The in vitro uptake of CF-CAPC encasing soy oil containing Nile red is shown for fixed CHO cells (FIG. 11). The cofocal images show uptake after 2 hr. The carboxyfluorescein label (top left) is seen throughout the cytoplasm with some concentration in the perinuclear region. The DAPI stained cell nuclei are shown (top center). The Nile red soy oil mixture displays a similar cellular distribution as the CF-labeled peptide (top right). The bright field image and the merge of the three fluorophors are shown (bottom left and bottom center, respectively). Bottom right is blank. From this uptake experiment it is not possible to discern any breakdown of the CAPC with release of the oil. From other initial in vitro experiments, when CAPC containing soy oil are used as controls we see growth enhancement of the cells suggesting that the nitrogen rich amino acids and the oils are being released and metabolized (data not shown).

### Conclusion

CAPC encase low dielectric oils and solvents, generating colloidal suspensions in water that range in size from tens of nanometers to microns. These particles are positively charged and are easily resized using polycarbonate sizing filters. In the case of coconut oil, much of the oil retains the physical characteristics of the pure solvent when encapsulated. The CAPC are heat stable to 70° C. and at RT are stable for more than a year. The cationic nature of their surface facilitates their uptake by cells in culture. The lysine residues on their surface can be chemically modified for targeting purposes. From a drug delivery standpoint, encapsulating hydrophobic drugs within a cell-targeting CAPC prevent dilution of the active ingredient in the blood stream.

### Example 2

### General Protocol for CAPC Formation

### 2 mg of Peptide to Encapsulate 10 μL of Lipid

Pipette solution containing 2 mg of peptide into an Eppendorf tube and dry using, e.g., speed vac centrifuge (depending on the concentration of peptide and liquid volume, this may take anywhere from 30 minutes to a couple of hours). The dried tube should be at room temperature with a slight film on the bottom of the tube. Add 10 μL of desired lipid or oil to the dried peptide in the Eppendorf tube and vortex for 30 seconds to intermix the peptide and lipid. Add 990 μL distilled water to the tube, and place the tube into a water bath sonicator at 37 kHz, 100% for 30 minutes to one hour. The water bath can start at room temperature. Typically, within 30 minutes, the solution in the tube should turn to a milky white uniform suspension as the colloidal particles form around and sequester the lipid droplets. The suspension should be sonicated until it appears homogenous or uniform, and can be subjected to sonication for another 15 minutes to 30 minutes at the same power under the uniform suspension of colloids forms. The suspension is then subjected to DLS and Zeta analysis for quality control purposes.

### Example 3

### Effect of Sonication Time

To ascertain the optimum period for CAPC formation by sonication, a solution containing—water (40 mL), peptide (5 mg) and oil (50 μL) were added together and sonicated for 10 sec before initial reading was taken. The sample was then sonicated continuously for 30 min on a Helios Br Laser diffraction instrument (Sympatec GmbH, Clausthal-Zellerfeld, Germany) with a built-in probe sonicator. Size scans were recorded every 3 min.

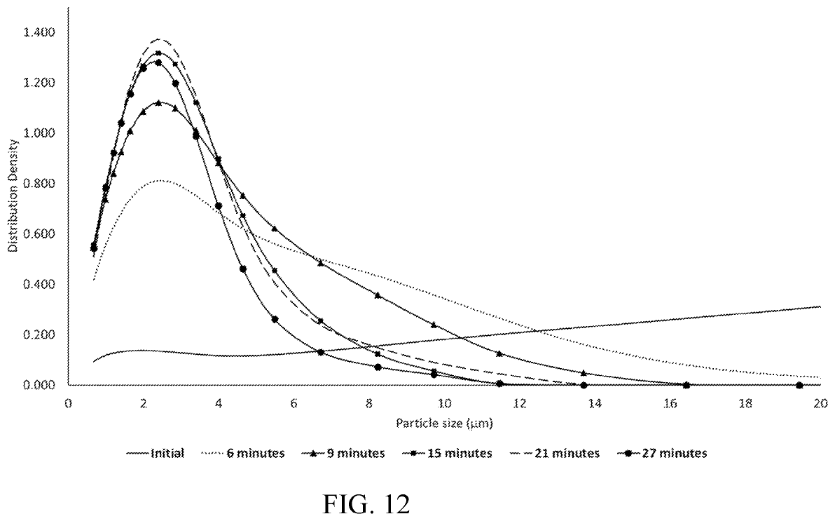

The scan recorded after the initial 10 sec pulse does not reveal any micron sized particles. As shown in FIG. 12, it is not until 6 min that a wide distribution of particles ranging from the lower instrument detection limit of 1 micron to greater than 16 microns are observed. At each subsequent time point up to 21 min the particle sizes decrease, and the population becomes more homogeneous. At 27 min a slight decrease in the distribution density is observed, however the homogeneity is improving. This experiment was repeated four times with nearly identical results obtained. Based on this study a sonication time of 30 min was adopted for all subsequent experiments.

### Example 4

### Colloidal Particles Encapsulating Cannabidiol (CBD)

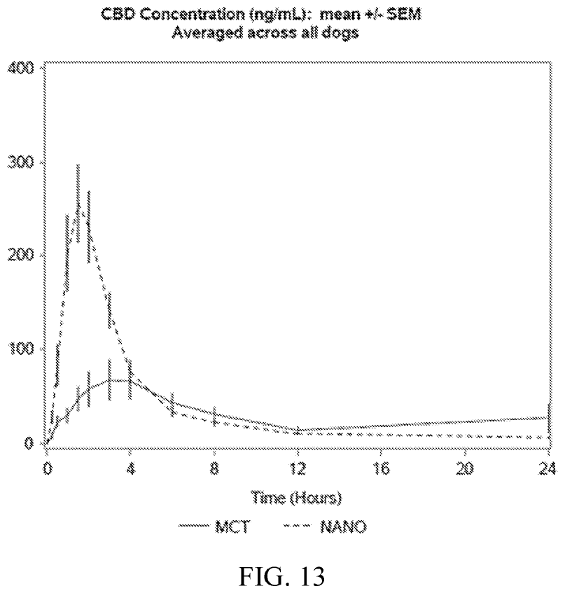

Cannabidiol (CBD) is a notoriously difficult active compound to encapsulate and deliver. It is a BCS Class II drug with poor solubility and poor bioavailability. This study was a comparative pharmacokinetic (PK) study utilizing two groups of seven dogs per group. In this Example, CBD was formulated into CAPCs and also compared to MCT Oil as a negative control. The CAPC nano-CBD formulation was a solution containing—water, CAPC peptide and Soy oil with dissolved CBD mixed together and sonicated continuously for 30 min. A total of 14 dogs were randomized to one of two treatment groups: “NANO” for those treated with the nanoformulated CAPCs or “MCT” for those in the control group. Dogs were dosed orally with either formulation. For Treatment Period I, Group 1 animals were treated with CBD-MCT (2 mg/Kg) and Group 2 animals were treated with CBD-NANO (2 mg/Kg). Blood samples were collected prior to dosing (0) and at 15 min., 30 min. and 1, 1.5, 2, 3, 4, 5, 6, 8, 12, and 24 hours after treatment. Plasma was analyzed for CBD concentrations utilizing a qualified analytical method validated for canine plasma. The results are shown in FIG. 13. NANO versus MCT values were significantly different at P<0.05 (only AUC_{0-LOQ} and Cₘₐₓ were subject to statistical analysis). AUC_{0-LOQ} and Cₘₐₓ values were In transformed prior to analysis. Back-transformed least squares means were as follows: MCT: AUC_{0-LOQ} and Cₘₐₓ: 428 hr*ng/ml and 54 ng/ml, respectively. NANO: AUC_{0-LOQ} and Cₘₐₓ: 890 hr*ng/ml and 260 ng/ml, respectively. CAPC nano-CBD formulation as compared to a conventional MTC oil formulation resulted in a significantly increased CBD bioavailability when orally dosed in dogs.

1. A dried composition comprising a plurality of lyophilized or spray-dried peptide-based colloidal particles, each comprising a peptide monolayer encapsulating a droplet of non-polar excipient, optionally along with one or more hydrophobic and/or poorly water-soluble active agents dispersed or distributed therein, wherein said peptide monolayer comprises a plurality of amphipathic linear peptides having an N-terminal hydrophobic segment and a C-terminal hydrophilic segment.

2. A kit comprising a unit dosage form of the dried composition of claim 1 in a container; and instructions for rehydrating said composition using an aqueous solvent system.

3. A method of preparing a suspension of colloidal particles, the method comprising:
mixing a dried composition according to claim 1 with an aqueous solvent system to rehydrate said lyophilized or spray-dried peptide-based colloidal particles, wherein said rehydrated peptide-based colloidal particles remain uniformly suspended in said aqueous solvent system, and wherein said non-polar excipient remains encapsulated in said peptide monolayer of each particle after said rehydrating step.

4. A method of resizing peptide-based colloidal particles, the method comprising:
providing a suspension of colloidal particles having a first average diameter, each comprising a peptide monolayer encapsulating a droplet of non-polar excipient, optionally along with one or more hydrophobic and/or poorly water-soluble active agents dispersed or distributed therein, wherein said peptide monolayer comprises a plurality of amphipathic linear peptides having an N-terminal hydrophobic segment and a C-terminal hydrophilic segment;
extruding said suspension of colloidal particles through an opening having a dimension smaller than said first average diameter; and
collecting said extruded colloidal particles, wherein said extruded colloidal particles have a second average diameter that is smaller than said first average diameter.

5. The method of claim 4, wherein said extruding step comprises passing said suspension of colloidal particles through a bore opening of a syringe and/or through a pore of a filter.

6. The method of claim 4, wherein said second average diameter is 200 nm or less.

7. A compositionaccording to claim 1, wherein said non-polar excipient is a lipid, fat, grease, wax, or oil.

8. A composition according to claim 1, wherein said non-polar excipient is a low dielectric solvent selected from the group consisting of alkanes (pentane, hexane, heptane, and n-Decane), cycloalkanes (cyclohexane), diethyl ether, carbon tetrachloride, methylene chloride, aromatics (benzene, toluene, and xylene), phthalate esters (diethyl phthalate, piperonyl butoxide, and combinations thereof.

9. A composition comprising a plurality of peptide-based colloidal particles, each comprising a peptide monolayer encapsulating a room temperature solid lipid, fat, grease, wax, or oil, optionally along with one or more hydrophobic and/or poorly water-soluble active agents dispersed or distributed therein, wherein said peptide monolayer comprises a plurality of amphipathic linear peptides having an N-terminal hydrophobic segment and a C-terminal hydrophilic segment.

10. The composition of claim 9, wherein said room temperature solid lipid, fat, grease, wax, or oil is selected from the group consisting of coconut oil, stearate-based glycerols, triglycerides, partial glycerides, fatty acids, steroids, and waxes.

11. A method of preparing a composition according to claim 9, the method comprising:
heating a room temperature solid lipid, fat, grease, wax, or oil to a temperature above its melting point, wherein the room temperature solid lipid, fat, grease, wax, or oil transitions to a liquid state;
optionally dissolving said one or more hydrophobic and/or poorly water-soluble active agents in said liquid state lipid, fat, grease, wax, or oil;
dispersing a plurality of said amphipathic peptides into said liquid state lipid, fat, grease, wax, or oil to create a mixture;
adding an aqueous solvent system to said mixture and agitating for a sufficient period of time to yield a suspension of peptide-based colloidal particles in said aqueous solvent system; and
cooling said suspension to at least room temperature,
wherein said peptide-based colloidal particles, after said cooling, comprise said peptide monolayer encapsulating solidified lipid, fat, grease, wax, or oil, optionally with said one or more hydrophobic and/or poorly water-soluble active agents, if present, dispersed or distributed therein.

12. A composition according to claim 1, wherein said amphipathic linear peptides having an N-terminal hydrophobic segment and a C-terminal hydrophilic segment have an overall chain length of 5 to 20 amino acid residues.

13. A composition according to claim 1, wherein said amphipathic linear peptides having an N-terminal hydrophobic segment and a C-terminal hydrophilic segment each comprise a hydrophobic segment comprising from about 3 residues to about 11 residues in length.

14. A composition according to claim 1, wherein said amphipathic linear peptides having an N-terminal hydrophobic segment and a C-terminal hydrophilic segment each comprise a hydrophobic segment comprising at least one phenylalanine residue, and at least two or more additional residues selected from the following residues: leucine, isoleucine, and/or valine.

15. A composition according to claim 1, wherein said amphipathic linear peptides having an N-terminal hydrophobic segment and a C-terminal hydrophilic segment each comprise a hydrophilic segment comprising from about 1 to about 7 hydrophilic amino acid residues, preferably lysine.


SEQUENCE LISTING (36 sequences)

SEQ ID NO: 1 - protein, 5 aa, synthetic construct
  VARIANT 1: note = X is F or V
XLIVI

SEQ ID NO: 2 - protein, 9 aa, synthetic construct
  VARIANT 1: note = X is F or V
XLIVIGSII

SEQ ID NO: 3 - protein, 7 aa, synthetic construct
  VARIANT 1: note = X is F or V
XFFIVIL

SEQ ID NO: 4 - protein, 12 aa, synthetic construct
  VARIANT 1: note = X is F or V
XLIVIGSIIVIL

SEQ ID NO: 5 - protein, 5 aa, synthetic construct
KKKKK

SEQ ID NO: 6 - protein, 10 aa, synthetic construct
FLIVIKKKKK

SEQ ID NO: 7 - protein, 14 aa, synthetic construct
FLIVIGSIIKKKKK

SEQ ID NO: 8 - protein, 11 aa, synthetic construct
FLIVIKKKKKC

SEQ ID NO: 9 - protein, 10 aa, synthetic construct
IVFLIKKKKK

SEQ ID NO: 10 - protein, 10 aa, synthetic construct
FLLLLKKKKK

SEQ ID NO: 11 - protein, 10 aa, synthetic construct
FAAAAKKKKK

SEQ ID NO: 12 - protein, 7 aa, synthetic construct
VFFIVIL

SEQ ID NO: 13 - protein, 12 aa, synthetic construct
FLIVIGSIIVIL

SEQ ID NO: 14 - protein, 5 aa, synthetic construct
  VARIANT 1: note = X is F or V
XLIIV

SEQ ID NO: 15 - protein, 5 aa, synthetic construct
  VARIANT 1: note = X is F or V
XLVII

SEQ ID NO: 16 - protein, 5 aa, synthetic construct
  VARIANT 1: note = X is F or V
XIVIL

SEQ ID NO: 17 - protein, 5 aa, synthetic construct
  VARIANT 1: note = X is F or V
XIIVL

SEQ ID NO: 18 - protein, 5 aa, synthetic construct
  VARIANT 1: note = X is F or V
XILVI

SEQ ID NO: 19 - protein, 5 aa, synthetic construct
  VARIANT 1: note = X is F or V
XVIIL

SEQ ID NO: 20 - protein, 5 aa, synthetic construct
  VARIANT 1: note = X is F or V
XVLII

SEQ ID NO: 21 - protein, 5 aa, synthetic construct
  VARIANT 1: note = X is F or V
XVILI

SEQ ID NO: 22 - protein, 5 aa, synthetic construct
  VARIANT 5: note = X is F or V
LIVIX

SEQ ID NO: 23 - protein, 5 aa, synthetic construct
  VARIANT 4: note = X is F or V
IVIXL

SEQ ID NO: 24 - protein, 5 aa, synthetic construct
  VARIANT 3: note = X is F or V
VIXLI

SEQ ID NO: 25 - protein, 5 aa, synthetic construct
  VARIANT 2: note = X is F or V
IXLIV

SEQ ID NO: 26 - protein, 5 aa, synthetic construct
  VARIANT 5: note = X is F or V
LIIVX

SEQ ID NO: 27 - protein, 5 aa, synthetic construct
IVFLI

SEQ ID NO: 28 - protein, 5 aa, synthetic construct
IIVLF

SEQ ID NO: 29 - protein, 5 aa, synthetic construct
VFLII

SEQ ID NO: 30 - protein, 5 aa, synthetic construct
IIVFL

SEQ ID NO: 31 - protein, 5 aa, synthetic construct
LIVIF

SEQ ID NO: 32 - protein, 5 aa, synthetic construct
IVILF

SEQ ID NO: 33 - protein, 9 aa, synthetic construct
FLIVIKKKK

SEQ ID NO: 34 - protein, 8 aa, synthetic construct
FLIVIKKK

SEQ ID NO: 35 - protein, 7 aa, synthetic construct
FLIVIKK

SEQ ID NO: 36 - protein, 6 aa, synthetic construct
FLIVIK